(19) United States
(12) Patent Application Publication
(10) Pub. No.: US 2026/0240297 A1
(43) Pub. Date: Aug. 20, 2026

(54) UV Light Embedded Makeup Case

(71) Applicant: John L. Castro, Palm Bay, FL (US)

(72) Inventor: John L. Castro, Palm Bay, FL (US)

(21) Appl. No.: 19/541,311

(22) Filed: Feb. 16, 2026

Related U.S. Application Data
(60) Provisional application No. 63/759,270, filed on Feb. 17, 2025.

(51) Int. Cl. A45C 15/06; A45C 11/00; A45C 15/04; A61L 2/104; A61L 103/45

(52) U.S. Cl. CPC A45C 15/06; A45C 11/008; A45C 15/04; A61L 2/104; A61L 2103/45; A61L 2202/11; A61L 2202/122; A61L 2202/16

(57) ABSTRACT

A UV light embedded makeup case including a first half with a motherboard having a plurality of ultraviolet lights and a second half with a plurality of magnets. It includes at least one mirrored panel with a plurality of panel holes. The panel holes are positioned to align with both the plurality of ultraviolet lights in the first half and the plurality of magnets in the second half when both the first and second halves are fitted together. The mirrored panel is structured to cover the motherboard and the UV light embedded makeup case is configured to project ultraviolet light within the makeup case to sanitize makeup stored therein.

### FIELD OF THE INVENTION

The present invention relates to makeup cases. In particular, the present invention relates to an ultraviolet light embedded makeup case.

### BACKGROUND

Up to 90% of makeup containers are contaminated with dangerous bacteria like E. coli, Staph, and Salmonella due to expired products and poor cleaning, often causing infections like pink eye, skin rashes, and impetigo. Improperly stored products create ideal breeding grounds for germs. Eye infections from makeup, such as bacterial conjunctivitis, styes, and keratitis, often result from poor hygiene, causing symptoms like swelling, itching, and discharge.

There is a need to safely store and disinfect makeup at the same time. Therefore, there exists a need for a UV light embedded makeup case as described and claimed herein.

### SUMMARY

A UV light embedded makeup case including a first half with a motherboard having a plurality of ultraviolet lights and a second half with a plurality of magnets. It may include at least one mirrored panel with a plurality of panel holes. The panel holes may be positioned to align with both the plurality of ultraviolet lights in the first half and the plurality of magnets in the second half when both the first and second halves are fitted together. The mirrored panel may be structured to cover the motherboard and the UV light embedded makeup case may be structured to project ultraviolet light within the makeup case to sanitize makeup stored therein.

The first half may be structured to fit into place with the second half to create a storage case. The second half may include makeup storage spaces with makeup therein and the ultraviolet lights may be structured to be off when the first half is positioned at an inactive distance. The ultraviolet lights may be structured to be on when the first half is positioned at an active distance. Likewise, the ultraviolet light may include a wavelength range short enough to disinfect its covered area, which may be between 254-275 nanometers.

Another embodiment may include a UV light embedded makeup case with a first half comprising a motherboard with a plurality of ultraviolet lights, a mirrored panel fitted within the first half comprising a plurality of panel holes, and a second half with a plurality of magnets and a second half mirrored panel with panel holes. The panel holes may be positioned to align with both the plurality of ultraviolet lights in the first half and the plurality of magnets in the second half when both the first and second halves are fitted together. The mirrored panel may be structured to cover the motherboard and may be structured to project ultraviolet light within the makeup case when closed to sanitize makeup stored therein.

The mirrored panel within the first half and the mirrored panel within the second half may be structured to reflect distributed light to spread throughout the inside of the case when the ultraviolet lights are active and on. The second half may include makeup storage spaces with makeup and makeup utensils therein.

The ultraviolet lights may be structured to be off when the first half is positioned at an inactive distance and on when the first half is positioned at an active distance. It may have ultraviolet light with a wavelength range short enough to disinfect its covered area, which may be a wavelength range between 254-275 nanometers. The first and second halves may combine to form the makeup case with the first half fitting into the second half.

In another embodiment there may be a UV light embedded makeup case with a first half having a motherboard with at least one ultraviolet light, a transparent panel fitted within the first half, and a second half with a plurality of spaces sized to accommodate a variety of different makeup and makeup utensils. The case may be structured to project ultraviolet light through the transparent panel within the makeup case when closed to sanitize makeup stored therein. In this embodiment, the transparent panel may be opaque. Furthermore, the at least one ultraviolet light may be a plurality of LEDs.

### BRIEF DESCRIPTION OF THE DRAWINGS

FIG. 1 is an exploded side perspective view of the UV light embedded makeup case according to an embodiment of the invention.

FIG. 2A is a demonstrative view of the UV light embedded makeup case according to an embodiment of the invention.

FIG. 2B is a demonstrative view of the UV light embedded makeup case according to an embodiment of the invention.

FIG. 3 is an exploded side perspective view of the UV light embedded makeup case according to an embodiment of the invention.

FIG. 4 is a side perspective view of the UV light embedded makeup case according to an embodiment of the invention.

FIG. 5 is a side perspective environmental view of the UV light embedded makeup case according to an embodiment of the invention in an open position.

FIG. 6 is a side perspective view of the UV light embedded makeup case according to an embodiment of the invention in a closed position.

FIG. 7 is a is a demonstrative view of the UV light embedded makeup case according to an embodiment of the invention.

FIG. 8 is a demonstrative view of the UV light embedded makeup case according to an embodiment of the invention.

FIG. 9 is a top view of a component of the UV light embedded makeup case according to an embodiment of the invention.

FIG. 10 is a circuit diagram of the component illustrated in FIG. 9 of the UV light embedded makeup case.

FIG. 11 is a side perspective view of the UV light embedded makeup case according to an embodiment of the invention.

FIG. 12 is a side perspective view of the UV light embedded makeup case according to an embodiment of the invention.

FIG. 13 is a side perspective view of the UV light embedded makeup case according to an embodiment of the invention.

FIG. 14 is a side perspective view of the UV light embedded makeup case according to an embodiment of the invention.

### DETAILED DESCRIPTION OF THE INVENTION

The present invention will now be described in detail with reference to the accompanying drawings. The embodiment descriptions are illustrative and not intended to be limiting in any way. Other embodiments of the invention will readily suggest themselves to persons with ordinary skill in the art after having the benefit of this disclosure. Accordingly, the following embodiments are set forth without any loss of generality and without imposing limitation upon the claimed invention.

Directional terms such as “above” “below” “upper” “lower” and other like terms are used for the convenience of the reader in reference to the drawings. Additionally, the description may contain terminology to convey position, orientation, and direction without departing from the principles of the present invention. Such positional language should be taken in context of the represented drawings.

Quantitative terms such as “generally” “substantially” “mostly” and other like terms are used to mean that the referred object, characteristic, or quality constitutes a majority of the referenced subject. Likewise, use of the terms such as first and second do not necessarily designate a limitation of quantity. Such terms may be used as a method of describing the presence of at least one of the referenced elements or may provide a means of differentiating orientation. The meaning of any term within this description is dependent upon the context within which it is used, and the meaning may be expressly modified.

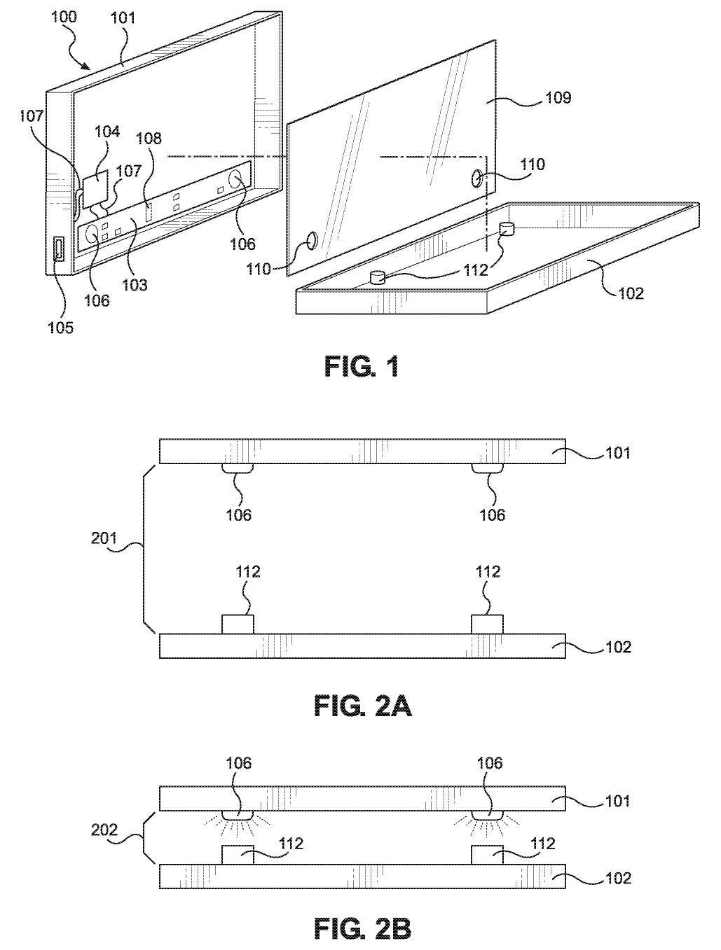

Referring now to FIG. 1, the UV light embedded makeup case 100, hereinafter referred to as the case 100, may include a first half 101, a second half 102, and at least one mirrored panel 109. Within the first half 101 may be a motherboard 103 with a plurality of lights 106. In some embodiments, this may be a pair of LEDs 106 positioned proximate longitudinal ends of the motherboard 103. The motherboard 103 may include a controller 108 and may be electrically connected via circuitry 107 to a battery 104. The battery 104 may be electrically connected via circuitry 107 to a charging port located on an outside edge of the first half 101.

The second half 102 may include a plurality of magnets 112 positioned therein. In some embodiments, the case 100 may be rectangular, square, or circular whereby the first half 101 fits into place with the second half 102 to create a storage case. Furthermore, in some embodiments, the mirrored panel 109 may be congruent with, and fit inside the first half 101 and cover the motherboard 103. Furthermore, the mirrored panel 109 may include a plurality of panel holes 110 positioned to align with both the plurality of LEDs 106 in the first half 101 and the plurality of magnets 112 within the second half 102 when both the first and second halves 101, 102 are fitted together.

FIGS. 2A and 2B illustrate how the case 100 may actuate the LEDs 106. As shown, the LEDs 106 are in the “off” or inactive state when the first half 101 is positioned at an inactive distance 201 from the second half 102 and the magnets 102. However, when the first half 101 is positioned within a particular range of the second half magnets 112, defined as an active distance 202, the LEDs 106 become active and switch to an “on” state. The lights or LEDs 106 may emit ultraviolet light with a wavelength range short enough to disinfect its covered area. This may be UV-C light emitting a wavelength range between 254-275 nanometers (nm). This light may shine through the panel holes 110 to reach the second half 102. Additionally, the panel holes 110 may facilitate the reach of the magnetic field to the motherboard 103 and the LEDs 106. Furthermore, in some embodiments, the LEDs 106 may be programmed by the controller 108 to only be activated for a set period of time before turning off or becoming inactive even when they are within the active distance 202.

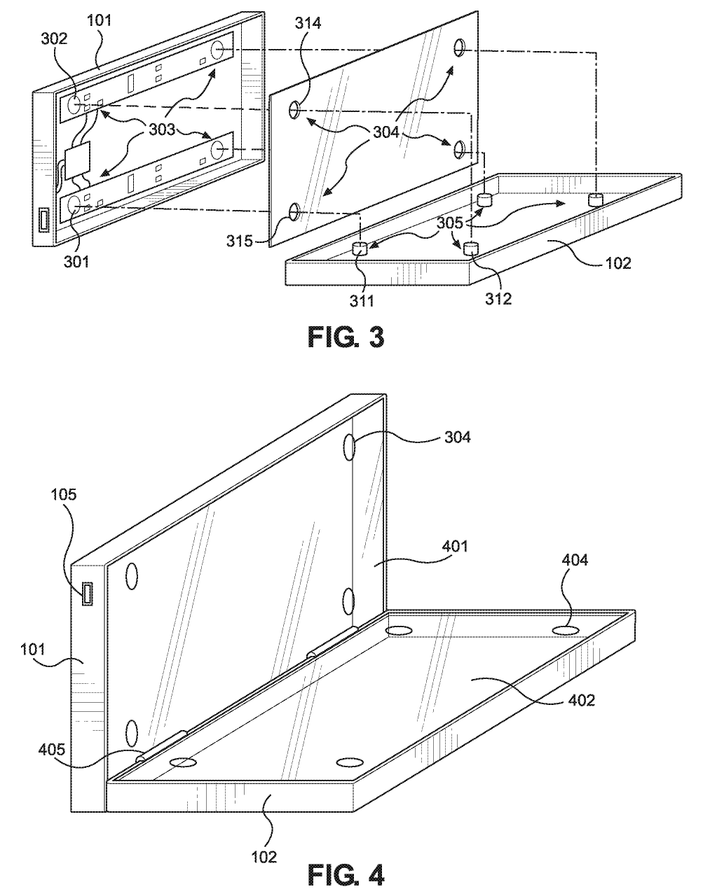

FIG. 3 illustrates a case 100 with a plurality of motherboards 103 and thereby a plurality of LEDs 106. In this embodiment, the motherboards 103 are set to position a plurality of LEDs 303 proximate the four corners of the first half 101. Therefore, there may be a first pair of LEDs 301 and a second pair of LEDs 302. Likewise, there may be a plurality of panel holes 304 including a first pair of panel holes 314 and a second pair of panel holes 315 positioned to align with the first and second pairs of LEDs 301, 302. Similarly, the second half may include a plurality of magnets 305 including a first pair of magnets 311 and a second pair of magnets 311 positioned within the second half 102 to align with the plurality of panel holes 314 and plurality of LEDs 303.

FIG. 4 illustrates an embodiment of the case 100 whereby the first half 101 is fitted together with the second half 102 via a pair of hinges 405. Therefore, the first half 101 may removably fold on top of the second half 102 to open and shut the case. Furthermore, the case is shown with a second half mirrored panel 402 with a plurality of panel holes 404 corresponding to the positioning of the plurality of magnets 305 underneath. Likewise, the first half mirrored panel 401 may include a plurality of panel holes 304 corresponding to the positioning of the plurality of LEDs 303 underneath.

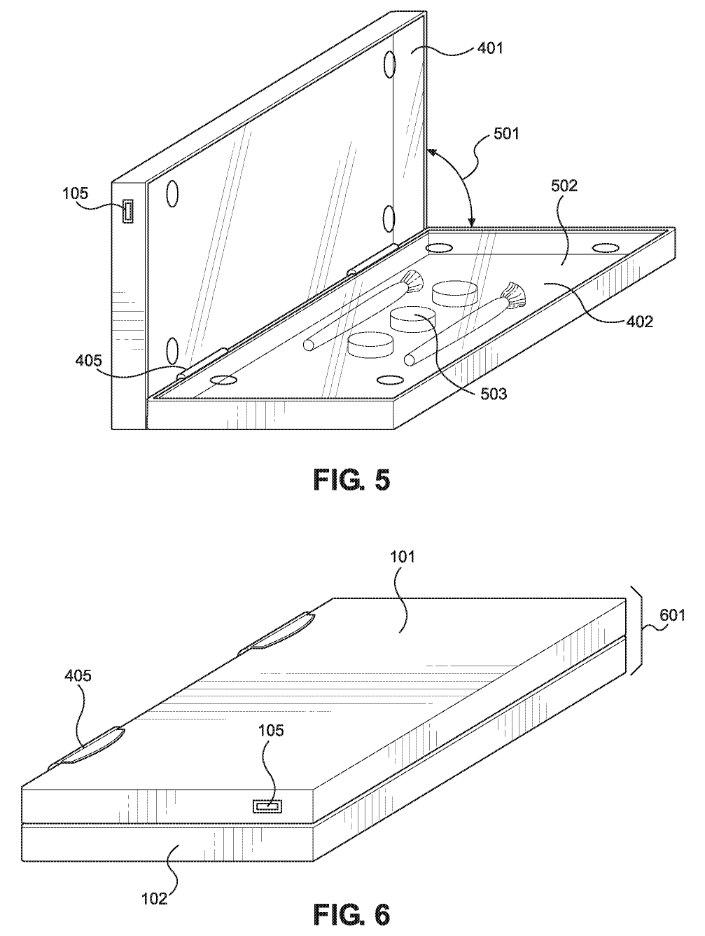

FIG. 5 illustrates how the case 100 may store makeup utensils 503 within a storage cavity 502 inside the case 100. In this figure, the case 100 is shown in the open position 501 and the makeup utensils 503 are shown resting atop the second mirrored panel 402. Also shown, is the charging port 105 placed in a different position than in the other figures emphasizing that the placement of the charging port 105 may be changed depending on need, preference, and circumstance.

FIG. 6 illustrates the case 100 in a closed position 601 whereby the first half 101 has been rotated and positioned on top of the second half 102 via the hinges 405. Here the LEDs 106 are placed in the “on” state because they are within the active distance 202 of the second half magnets 112.

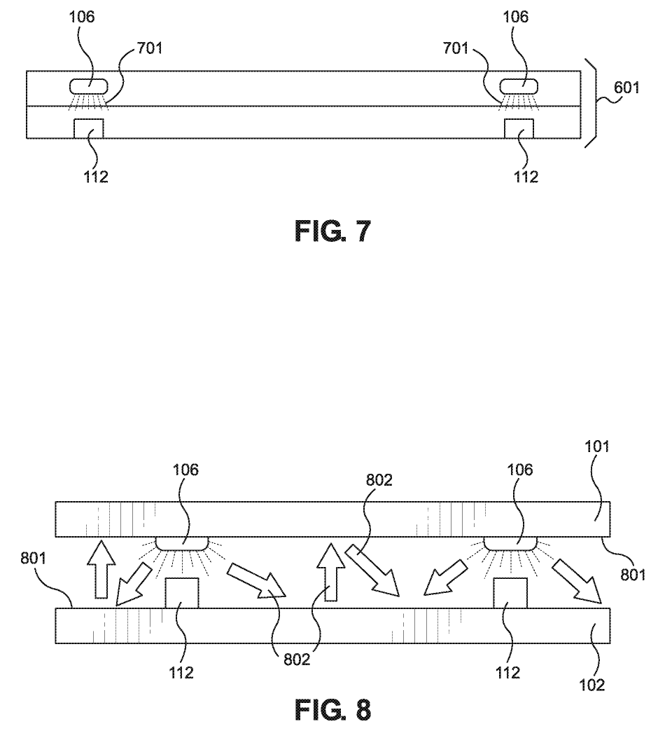

FIG. 7 shows that when the first half 101 is placed on top of the second half 102, the LEDs 106 are active and on and shining through to the second half 102 via passthrough light 701.

FIG. 8 illustrates that both the first half 101 and the second half 102 may include reflective surfaces 801 that may allow for distributed light 802 to spread throughout the inside of the case 100 when the LEDs 106 are active and on. The light from the LEDs 106 may pass through the plurality of panel holes 304 in the first half 101 and reflect off of the first and second mirrored panels 401, 402 to distribute UV light throughout the inside of the case 100 thereby disinfecting the makeup utensils 503 therein.

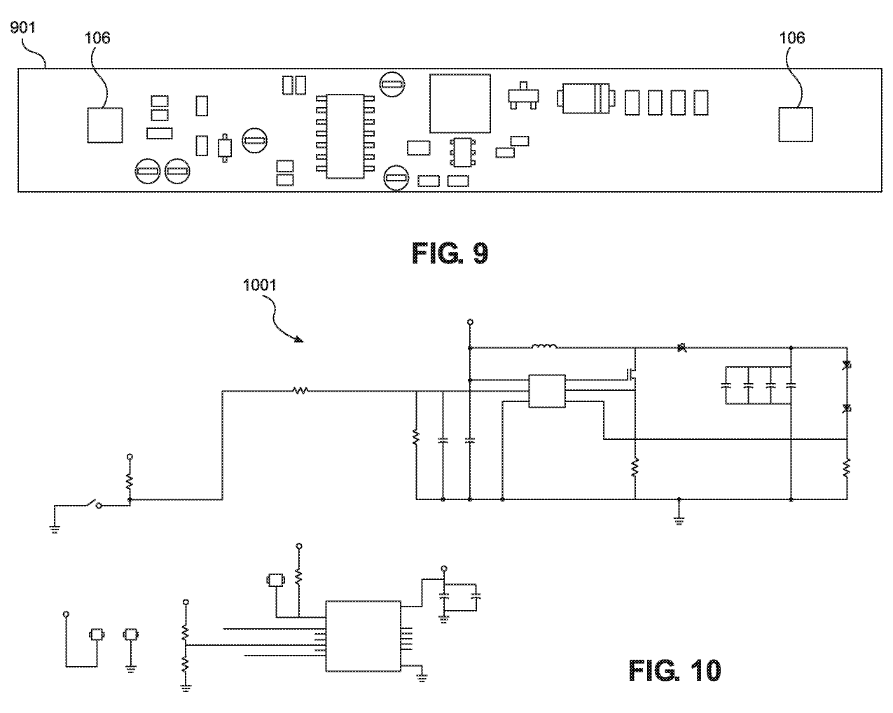

FIGS. 9 and 10 illustrate the motherboard 103 and its circuitry. FIG. 9 shows one potential arrangement of the motherboard 103 with LEDs 106 positioned at either end of a rectangular board. FIG. 10 is a circuit diagram of one potential layout of the LED 106 wiring functionality.

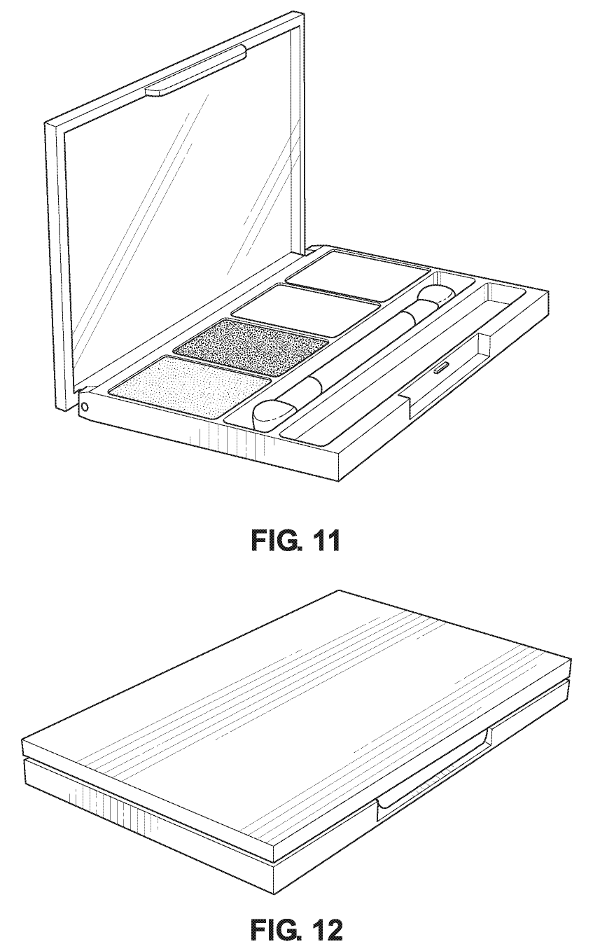

FIGS. 11, 12, 13 and 14 illustrate another embodiment whereby the first half 101 may include a transparent, translucent, or opaque panel instead of a mirror. In this embodiment, at least one ultraviolet light, or LED, or in some embodiments a plurality of ultraviolet lights or LEDs may shine through the panel to disinfect makeup contained in the second half 102. As shown, the second half 102 may include a plurality of spaces sized to accommodate a variety of different makeup and makeup utensils.

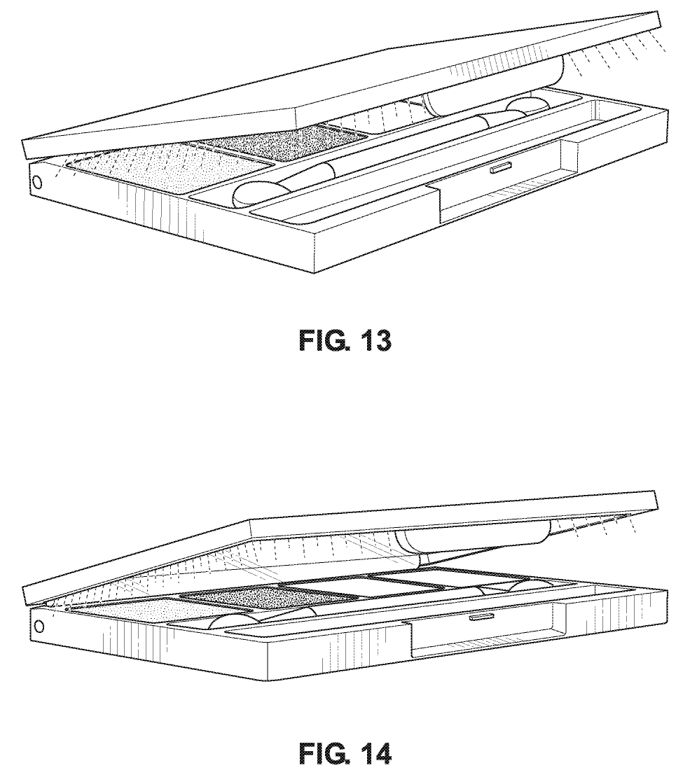

As shown by FIG. 11, when the case 100 is in an open position and at an inactive distance, the ultraviolet lights are not on. As shown by FIGS. 13 and 14, as the case 100 is being closed, and it reaches the active distance, the ultraviolet lights pass the active threshold distance and turn on. The ultraviolet lights may continue to be in an active on status while the case is closed. In some embodiments, that may be for a predetermined amount of time as programmed by an internal timer.

1. A UV light embedded makeup case comprising:
a first half comprising a motherboard with a plurality of ultraviolet lights;
a second half comprising a plurality of magnets;
at least one mirrored panel comprising a plurality of panel holes;
wherein the panel holes are positioned to align with both the plurality of ultraviolet lights in the first half and the plurality of magnets in the second half when both the first and second halves are fitted together;
wherein the mirrored panel is configured to cover the motherboard; and
wherein the UV light embedded makeup case is configured to project ultraviolet light within the makeup case to sanitize makeup stored therein.

2. The UV light embedded makeup case of claim 1 wherein the first half is configured to fit into place with the second half to create a storage case.

3. The UV light embedded makeup case of claim 1 wherein the second half comprises makeup storage spaces with makeup therein.

4. The UV light embedded makeup case of claim 1 wherein the ultraviolet lights are configured to be off when the first half is positioned at an inactive distance.

5. The UV light embedded makeup case of claim 1 wherein the ultraviolet lights are configured to be on when the first half is positioned at an active distance.

6. The UV light embedded makeup case of claim 1 wherein the ultraviolet light comprises a wavelength range short enough to disinfect its covered area.

7. The UV light embedded makeup case of claim 1 wherein the ultraviolet light comprises a wavelength range between 254-275 nanometers.

8. A UV light embedded makeup case comprising:
a first half comprising a motherboard with a plurality of ultraviolet lights;
a mirrored panel fitted with the first half comprising a plurality of panel holes;
a second half comprising a plurality of magnets and a second half mirrored panel with panel holes;
wherein the panel holes are positioned to align with both the plurality of ultraviolet lights in the first half and the plurality of magnets in the second half when both the first and second halves are fitted together;
wherein the mirrored panel is configured to cover the motherboard; and
wherein the UV light embedded makeup case is configured to project ultraviolet light within the makeup case when closed to sanitize makeup stored therein.

9. The UV light embedded makeup case of claim 8 wherein the mirrored panel within the first half and the mirrored panel within the second half are configured to reflect distributed light to spread throughout the inside of the case when the ultraviolet LEDs are active and on.

10. The UV light embedded makeup case of claim 8 wherein the second half comprises makeup storage spaces with makeup and makeup utensils therein.

11. The UV light embedded makeup case of claim 8 wherein the ultraviolet lights are configured to be off when the first half is positioned at an inactive distance.

12. The UV light embedded makeup case of claim 8 wherein the ultraviolet lights are configured to be on when the first half is positioned at an active distance.

13. The UV light embedded makeup case of claim 8 wherein the ultraviolet light comprises a wavelength range short enough to disinfect its covered area.

14. The UV light embedded makeup case of claim 8 wherein the ultraviolet light comprises a wavelength range between 254-275 nanometers.

15. The UV light embedded makeup case of claim 8 wherein the first half is configured to fit into place with the second half to create a storage case.

16. The UV light embedded makeup case of claim 8 wherein there is a charging port on the first half.

17. The UV light embedded makeup case of claim 8 wherein there is a charging port on the second half.

18. A UV light embedded makeup case comprising:
a first half comprising a motherboard with at least one ultraviolet light;
a transparent panel fitted within the first half;
a second half comprising a plurality of spaces sized to accommodate a variety of different makeup and makeup utensils; and
wherein the UV light embedded makeup case is configured to project ultraviolet light through the transparent panel within the makeup case when closed to sanitize makeup stored therein.

19. The UV light embedded makeup case of claim 18 wherein the transparent panel is opaque.

20. The UV light embedded makeup case of claim 18 wherein the at least one ultraviolet light is a plurality of LEDs.
